(19) United States
(12) Patent Application Publication
(10) Pub. No.: US 2014/0316234 A1
(43) Pub. Date: Oct. 23, 2014

(54) APPARATUS AND METHODS FOR ACCURATE SURFACE MATCHING OF ANATOMY USING A PREDEFINED REGISTRATION PATH

(71) Applicant: PATHFINDER THERAPEUTICS, INC., Nashville, TN (US)

(72) Inventors: Jonathan Waite, Cary, NC (US); Brian Lennon, Nasvhille, TN (US); Michael James Bartelme, Fort Collins, CO (US); Rasool Khadem, Superior, CO (US)

(73) Assignee: PATHFINDER THERAPEUTICS, INC., Nashville, TN (US)

(21) Appl. No.: 14/184,211

(22) Filed: Feb. 19, 2014

Related U.S. Application Data
(60) Provisional application No. 61/766,453, filed on Feb. 19, 2013.
(60) Provisional application No. 61/767,494, filed on Feb. 21, 2013.

(51) Int. Cl. A61B 19/00; A61B 5/06

(52) U.S. Cl. CPC A61B 19/5225; A61B 5/061
USPC 600/407

(57) ABSTRACT

A method includes scanning a bodily tissue of a patient with an imaging device and prior to an interventional procedure to produce an image of a surface of an organ. At least a portion of a registration path associated with the organ is defined. The method further includes surgically exposing the organ and placing a probing instrument in contact with the organ at a starting point associated with the registration path and moving the probing instrument substantially along the registration path to define a registration surface of the organ. The method further includes mapping the registration surface of the organ to the image of the surface of the organ based at least in part on the registration path.

### CROSS-REFERENCE TO RELATED APPLICATION

This application claims priority to and the benefit of U.S. Provisional Patent Application No. 61/766,453, filed Feb. 19, 2013, and entitled “APPARATUS AND METHODS FOR ACCURATE SURFACE MATCHING OF ANATOMY USING A PREDEFINED REGISTRATION PATH,” which is incorporated herein by reference in its entirety.

This application claims priority to and the benefit of U.S. Provisional Patent Application No. 61/767,494, filed Feb. 21, 2013, and entitled “WINDOW MANAGER,” which is incorporated herein by reference in its entirety.

### BACKGROUND

The embodiments described herein relate to image-guided surgical techniques and more particularly apparatus and methods for accurate surface matching of anatomy using salient features.

Image-guided therapy (IGT), which is also often referred to as image-guided intervention (IGI), has gained widespread attention and clinical acceptance for use in localizing tumors in abdominal organs. Procedures that utilize IGT include, but are not limited to, tumor biopsy, ablation, and resection. IGT describes the interactive use of medical images, often taken preoperatively, during a percutaneous procedure, and is often referred to as a “global positioning system” (GPS) for interventional radiology. By way of analogy, in an automobile GPS, the current position of a vehicle is accurately localized or “registered” onto an electronic roadmap that is updated as the automobile moves. The driver can use the GPS as a guide to see where their vehicle is, where it has been, where it is headed, and a planned route with which to follow to arrive at a selected destination. IGT allows the physician to accomplish essentially the same thing with one or more tracked medical instruments on a 3-D “roadmap” of highly detailed tomographic medical images of the patient that are acquired during and/or before the interventional procedure. Often, the key to an IGT procedure is the accurate registration between real “patient” space (e.g., during a procedure) and medical image space (e.g., preoperatively collected).

In some IGT procedures, a 3D map or plan is created from the preoperative diagnostic images, possibly days before the actual procedure and in consultation with a variety of physicians in different disciplines. On the day of the percutaneous procedure, the position of the patient and the medical instruments are accurately localized or “registered” onto the preoperative images. As the physician moves the instrument, the precise location of its tip is updated on the 3-D images. The physician can then quickly follow a planned path to a selected destination (for example, a tumor or other lesion of interest). The exact location of the instrument is confirmed with a form of real-time imaging, including, but not limited to, intraoperative computerized tomography (CT), 2-D fluoroscopy, or ultrasonic (US) imaging.

In some instances, the registration of the pre-operative images to patient space process can employ non-tissue reference markers and/or skin fiducial markers. In such instances, radio opaque fiducial markers (also known as skin fiducial markers) are attached to the patient's abdomen and a full CT scan of the patient's abdomen is taken immediately before the procedure (also known as intra-procedural images). In this manner, a point-based registration process is used to achieve correspondence between the location of the fiducial markers on the abdomen and the corresponding location in the intra-procedural CT images. In other instances, pre-operative images can be registered to the patient space during the procedure by tracking one or more instruments inserted into the body of the patient using a CT scan, 2-D fluoroscopy, or ultrasonic imaging.

In such instances, the highly detailed diagnostic images are often not easily used during the interventional procedure. For example, the physicians may have limited or no access to detailed visualizations of lesions and vasculature and/or have limited or no time to create an ideal procedure plan. Furthermore, the patients are scanned at least twice (once for pre-procedural diagnostic images and a second time for the intra-procedural images), which increases their exposure to X-ray radiation. Therefore, it is desirable to use the high quality diagnostic CT or MRI medical images directly for percutaneous guidance by performing a registration using the images. Point-based registration techniques described above, however, are often not sufficiently accurate, thereby compromising the accuracy of guidance during interventional procedures.

In some instance, a registration process can use surfaces generated from pre-operative diagnostic images and surfaces obtained during surgical or interventional procedures. In such instances, “salient anatomical features” (anatomical regions that can be easily identified on the surfaces of the diagnostic images and the anatomical surfaces) can be used to perform a rigid surface-based registration to align the surfaces obtained during surgical or interventional procedures to the pre-operative surfaces. In some instances, a clinician manually establishes a starting point and an ending point (and a plurality of points therebetween) of salient anatomical features to perform the registration of the physical surfaces to the pre-operative surfaces. Such starting points and ending points, however, are often difficult to identify in a reliable manner, thereby compromising the accuracy of the registration.

Thus, a need exists for apparatus and methods to accurately perform registration using salient anatomical features using a predefined path for salient feature identification during an interventional procedure.

### SUMMARY

Apparatus and methods for accurate surface mapping using salient anatomical features are described herein. In some embodiments, a method includes scanning a bodily tissue of a patient with an imaging device prior to an interventional procedure to produce an image of an organ, including the surface of the organ. At least a portion of a registration path associated with the organ is defined. In other words, a predefined path is provided for a clinician to follow in order to properly register an intraoperative image. The method further includes surgically exposing the organ and placing a probing instrument in contact with the organ at a starting point associated with the registration path and moving the probing instrument substantially along the predefined registration path to define a registration surface of the organ. The method further includes mapping the registration surface of the organ to the image of the surface of the organ based at least in part on the registration path.

### BRIEF DESCRIPTION OF THE DRAWINGS

FIG. 1 is a schematic illustration of a system for surface matching anatomy using salient anatomical features according to an embodiment.

FIG. 2 is a flowchart illustrating a method of surface matching anatomy using salient anatomical features according to an embodiment.

FIGS. 3-6 illustrate various organs having salient anatomical features that can be used to facilitate a registration of a physical surface to a pre-operative surface according to various embodiments.

### DETAILED DESCRIPTION

Apparatus and methods for accurate surface mapping using salient anatomical features are described herein. In some embodiments, a method includes scanning a bodily tissue of a patient with an imaging device and prior to an interventional procedure, producing an image of a surface of an organ. At least a portion of a registration path associated with the organ is defined. The method further includes surgically exposing the organ and placing a probing instrument in contact with the organ at a starting point associated with the registration path and moving the probing instrument substantially along the predefined registration path to define a registration surface of the organ. The method further includes mapping the registration surface of the organ to the image of the surface of the organ based at least in part on the registration path.

In some instances, the embodiments described herein can provide a framework for registering intra-procedural surface images of an organ with surfaces extracted from pre-procedural image data (e.g., magnetic resonance imaging (MRI) or computed tomography (CT) volumes) for the purposes of providing image-guidance during percutaneous surgical procedures. Registration is a method of determining the mathematical relationship between two coordinate spaces and is a component in image-guided surgery (IGS) devices. The goal of IGS is to allow the clinician to interactively use high resolution, high contrast preprocedural tomographic image data within the intervention via overlay display of tracked surgical instrumentation.

In some instances, a set of anatomical landmarks (i.e., salient anatomical features) are identified in the preoperative image volume by the surgeon and a three-dimensional image coordinate system is recorded. In some instances, unique geometric features of an organ are used to identify the overall shape of the organ and/or a surface of the organ. As described in detail herein, a starting point of a registration path can be defined at or by a salient anatomical feature and can be used to register intraoperative surface data to the image surface data.

Intraoperative surface images can be acquired using laser range scanning (LRS) technology, manually with an optically tracked stylus or ablation instrument, or via any other imaging modality. The registration process is then used within an image-guidance system (e.g., an imaging device and one or more electronic processing devices) to provide the mathematical mapping required to interactively use the pre-procedural image data for guidance within the intervention. In addition to hardware that is capable of performing surface data acquisition during percutaneous procedures, an image guidance device using the methods and system described herein may provide guidance information via a software interface. For example, in some embodiments, a navigation software interface can be used to map the location of tracked percutaneous ablation instrumentation onto the pre-procedural tomographic data. In some embodiments, the system can be used to compute the mathematical transformation that allows for the display of the location of tracked instrumentation on the pre-procedural tomographic image data. Moreover, the devices and methods described herein can provide accurate surface registration in a relatively short amount of time to display the trajectory and device locations relative to targets planned prior to surgery. In particular, pre-procedural image data is used for guidance, which allows for pre-procedural planning and 3-D model generation.

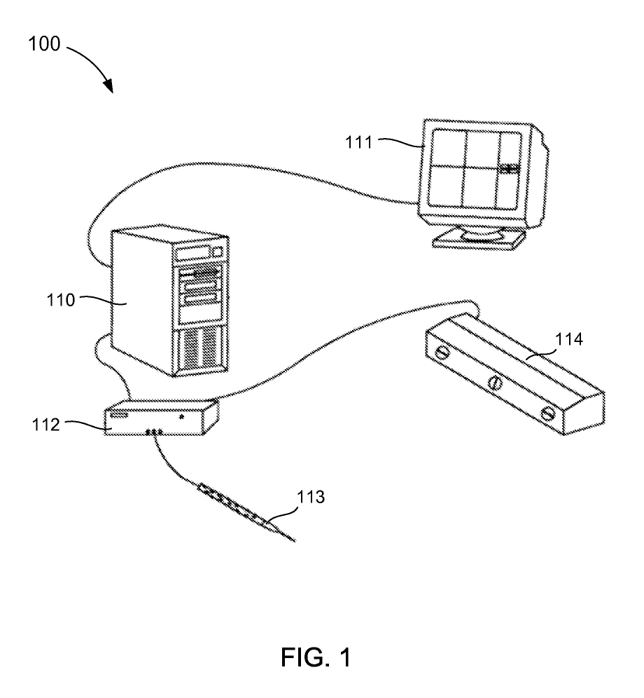

FIG. 1 is a schematic illustration of a system 100 for surface matching anatomy using salient anatomical features according to an embodiment. More particularly, the system 100 can be used in conjunction with preoperative images from an imaging process (e.g., a computerized tomography (CT) scan, 2-D fluoroscopy, ultrasonic (US) imaging, and/or magnetic resonance imaging (MRI), not shown in FIG. 1) to perform an image-guided interventional procedure such as a biopsy, ablation, resection, or the like. The system 100 includes at least an electronic processing device 110, a display 111, a controller 112, a probing instrument 113, and an optical tracking system 114.

The electronic processing device 110 can be, for example, a personal computer, or the like. The electronic processing device 110 includes at least a processor and a memory. The memory (not shown in FIG. 1) can be, for example, a random access memory (RAM), a memory buffer, a hard drive, a read-only memory (ROM), an erasable programmable read-only memory (EPROM), and/or so forth. In some embodiments, the memory of the electronic processing device 110 stores instructions to cause the processor to execute modules, processes, and/or functions associated with using a personal computer application, controlling one or more medical instruments, displaying and updating a medical image, and/or the like.

The processor (not shown in FIG. 1) of the electronic processing device 110 can be any suitable processing device configured to run and/or execute a set of instructions or code. For example, the processor can be a general purpose processor, a central processing unit (CPU), an accelerated processing unit (APU), or the like. In some embodiments, the processor of the electronic processing device 110 can be included in, for example, an application specific integrated circuit (ASIC). The processor can be configured to run and/or execute a set of instructions or code stored in the memory associated with using a personal computer application, a mobile application, an internet web browser, telephonic or cellular communication, and/or the like. More specifically, in some instances, the processor can execute a set of instructions or code stored in the memory associated with surface mapping anatomy using salient anatomical features. For example, the processor can execute a program for a window manager that assists with surface mapping anatomy, such as the window manager illustrated and described in U.S. Provisional Patent Application No. 61/767,494, which is incorporated by reference herein in its entirety.

The display 111 is in electronic communication with the electronic processing device 110. The display 111 can be any suitable display configured to provide a user interface to the electronic processing device 110. For example, the display 111 can be a cathode ray tube (CRT) monitor, a liquid crystal display (LCD) monitor, a light emitting diode (LED) monitor, and/or the like. The display 111 can be configured to provide the user interface for a personal computer application or the like. For example, the display 111 can be configured to graphically represent a medical image of an anatomical structure. In some embodiments, the display 111 can graphically represent the position of a medical instrument (e.g., the probing instrument 113, an ablation instrument, and/or any other suitable device) in contact with an organ or tissue relative to a preoperative image of the organ. Expanding further, in some embodiments, the processing device 110 can be configured to map a surface of the organ to a preoperative image of the organ and the display 111 can graphically represent a virtual position of the medical instrument relative to the image of the organ. For example, the display 111 can include a graphical user interface (GUI) that displays this graphical representation. The GUI can be part of a window manager, such as the window manager illustrated and described in U.S. Provisional Patent Application No. 61/767,494, which is incorporated by reference herein in its entirety.

As shown in FIG. 1, the electronic processing device 110 is in electronic communication with the controller 112 (e.g., via an Ethernet cable, universal serial bus (USB), SATA cable, eSATA cable, or the like). The controller 112 can be any suitable device for controlling at least a portion of the system 100. More specifically, the controller 112 can provide a user interface that can be manipulated by a user (e.g., a clinician, technician, doctor, physician, nurse, etc.) to control, for example, the probing instrument 113 and/or the optical tracking system 114.

The optical tracking sensor 114 can be, for example, an infrared tracking device. In some embodiments, the optical tracking sensor 114 can include any number of cylindrical lenses (e.g., three lenses) that can receive light from sequentially strobed infrared light emitting diodes (IREDs). In this manner, the optical tracking sensor 114 can triangulate to find each IRED relative to the position of the optical tracking sensor 114. In other embodiments, the optical tracking sensor 114 can be configured to sense a measure of reflected or refracted light. For example, in some embodiments, the optical tracking sensor 114 can broadcast an infrared light and can include one or more lenses configured to receive a portion of the infrared light that is reflected and/or refracted by a surface of the probing instrument 113 and/or an anatomical structure.

The probing instrument 113 can be any suitable instrument. For example, in some embodiments, the probing instrument 113 can include an ablation tip that can be used to microwave or heat-kill lesions. In some embodiments, the probing instrument 113 can include any number of IREDs that can be tracked by the optical tracking system 114. In this manner, the probing instrument 113 can be placed in contact with a surface of an organ to define registration points used to map the surface of the organ in physical space onto the surface of the organ in the preoperative (preop) image. More specifically, when a given number of IREDs are detected by the lenses of the optical tracking sensor 114, the tip of the probing instrument 113 and/or the registration point on the surface of the organ can be accurately localized in physical space without placing constraints on how the probing instrument 113 is handled by a surgeon.

In some embodiments, a probing instrument 113 can have 24 IREDs which spiral around the instrument's handle. In such embodiments, the probing instrument 113 can be sufficiently light to be easily directed and can be accurate with a tip location error of 0.35 mm in three-dimensional (3-D) space. In other embodiments, the probing instrument 113 can be formed from and/or include a surface configured to reflect a portion of light. For example, in some embodiments, the probing instrument 113 can reflect a portion of light broadcasted by the optical tracking sensor 114. In such embodiments, the optical tracking sensor 114 can receive at least a portion of the reflected light to determine the location of the probing instrument 113. Thus, in such embodiments, the probing instrument 113 need not include IREDs.

The probing instrument 113 can include any suitable activation portion configured to activate the probing instrument 113. For example, in some embodiments, the probing instrument 113 can be gesture activated. More specifically, the probing instrument 113 can be configured to emit a light from one or more of the IREDs based on the user making a specific gesture (e.g., moving, tilting, shaking, rotating, or otherwise reconfiguring the probing instrument 113). In other embodiments, the probing instrument 113 can include a push button, a switch, a toggle, a depressible tip, and/or any other suitable activation portion. Thus, the user can move the probing instrument 113 along a surface of the organ to register the surface relative to the preoperative image.

In some embodiments, the user can move the probing instrument 113 along a predefined path. For example, in some instances, a surgeon or clinician can define a starting point associated with a salient anatomical feature of an organ on a preoperative image and can define at least a portion of a path along the surface of the organ in the image. In such instances, during a procedure, the surgeon can locate the salient anatomical feature associated with the starting point and place the probing instrument 113 in contact with the surface of the organ in physical space associated with the starting point. The surgeon can move the probing instrument 113 along at least a portion of the predefined path associated with the surface of the organ in to the preoperative image. The optical tracking sensor 114 can track the probing instrument 113 and register position data associated with the probing instrument 113 relative to the surface of the organ in physical space and send a signal associated with the position data to the electronic processing device 110. Thus, the electronic processing device 110 can receive the signal and map the surface of the organ in physical space to the surface of the organ in the preoperative image. More specifically, by defining a starting point associated with a salient anatomical feature and by defining at least a portion of a path on the surface of the organ along which the probing instrument 113 is moved, the accuracy of the mapping can be increased and the time to determine the location of the registration points (e.g., on the physical surface of the organ) relative to preoperative image can be significantly decreased. In addition, by defining a starting point and at least a portion of the path, the surface of the organ can be determined algorithmically without registering substantially the entire surface of the organ.

In some embodiments, the probing instrument 113 can define a coordinate system in physical space and also preserves the registration point(s) if the patient is moved. For example in some embodiments, the system 100 can include a reference emitter (not shown) and the optical tracking sensor 114 can be configured to localize both the probing instrument 113 and the reference emitter in sensor unit space. By mapping the position of the probing instrument 320 into the space defined by the position and orientation of the reference emitter, the location of the optical tracking sensor 114 need not be identified during a registration (e.g., a mapping) process. Thus, the optical tracking sensor 114 can be flexibly placed before surgery and moved during the procedure to accommodate any surgical requirements.

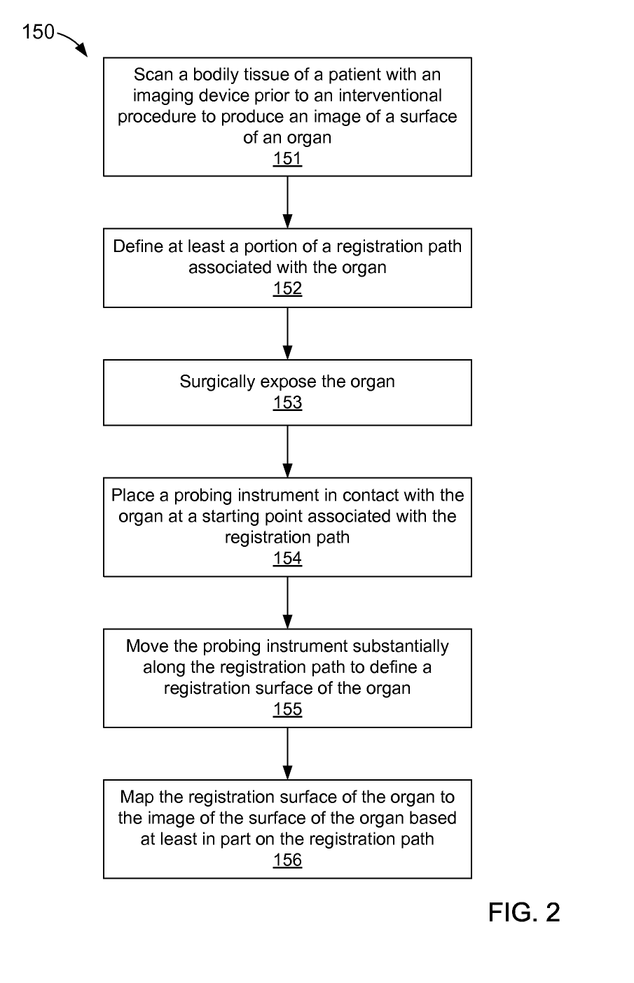

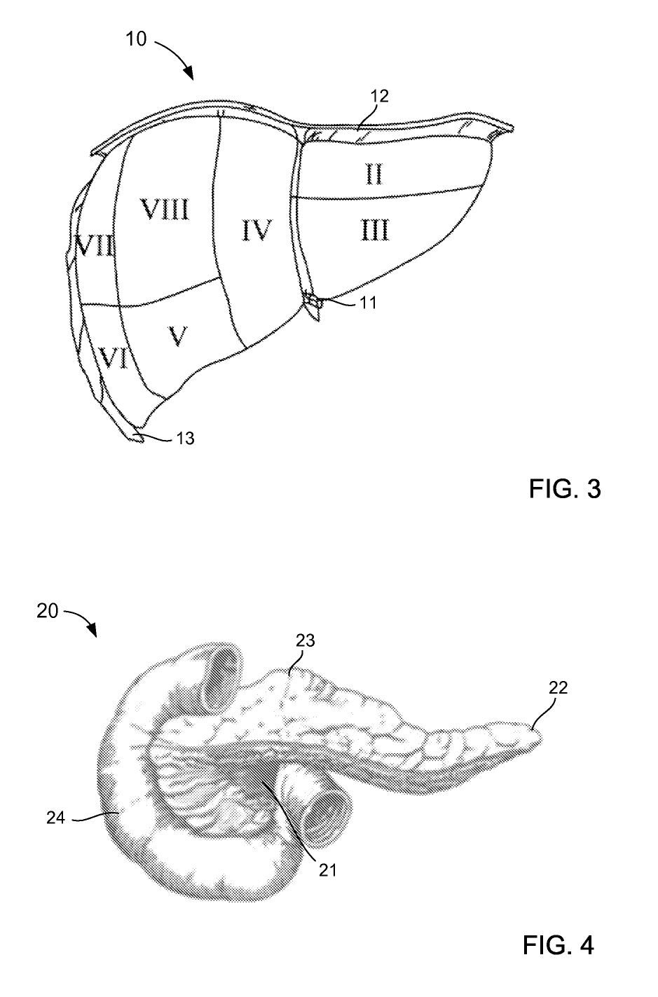

FIG. 2 is a flowchart illustrating a method 150 of surface matching anatomy using salient anatomical features according to an embodiment. In some instances, the method 150 can be used to map a surface of an organ in physical space (i.e., intraoperatively) to an image of the surface of the organ obtained preoperatively. Thus, the mapping of the surface of the organ in physical space onto the image of the surface of the organ can facilitate an image-guided interventional procedure such as, for example, a biopsy, ablation, and/or resection. The method 150 includes scanning a bodily tissue of a patient with an imaging device prior to an interventional procedure to produce an image of a surface of an organ, at 151. For example, in some instances, a portion of the patient can be medically imaged using a computerized tomography scan (CT), a magnetic resonance imaging scan (MRI), and/or an ultrasonic imaging scan (US). In some instances, the liver of the patient can be imaged and salient features of the liver can be identified. For example, as shown in FIG. 3, a liver 10 can be imaged and the falciform ligament 11, the left triangular ligament 12, and the right triangular ligament 13 can be identified. With the organ imaged, a user (e.g., a doctor, technician, physician, surgeon, nurse, etc.) can define at least a portion of a registration path associated with the organ, at 152. For example, in some instances, the base of the falciform ligament 11 can be identified on the image of the surface of the liver 10. In such instances, the user can define the registration path along the falciform ligament 11 in the superior direction to the left triangular ligament 12 and subsequently to the right triangular ligament 13.

In some embodiments, the user can manipulate an electronic device (e.g., the electronic processing device 110 shown in FIG. 1) to select and/or identify the starting point of the registration path. For example, in some embodiments, the user can engage an interactive touch screen or the like to select or identify the starting point of the registration path and/or the salient anatomical features. In some embodiments, an electronic device can be configured to store generic information associated with salient anatomical features (e.g., a global template or the like). In such embodiments, the electronic device can define surface data and/or salient anatomical features based at least in part on the surface curvature, surface shape, surface orientation, or the like. With the salient anatomical features identified and at least a portion of the registration path defined, the image of the surface of the organ (e.g., the liver 10) can be stored. Furthermore, by identifying the salient anatomical features and at least a portion of a registration path, the overall surface of the organ can be determined algorithmically, thereby reducing user interaction time. In some instances, with the organ imaged, the surgeon can virtually perform the procedure using the image of the organ, thereby increasing a success rate of the interventional procedure as well as reducing the duration of the procedure.

At 153, the organ can be surgically exposed during the interventional procedure. For example, the abdomen can be surgically opened to expose the liver 10. With the organ exposed, a probing instrument (e.g., the probing instrument 113 described with reference to FIG. 1) is placed in contact with the organ at the starting point (e.g., at a salient anatomical feature) associated with the registration path, at 154. For example, in some instances, the probing instrument can be placed in contact with the base of the falciform ligament 11 of the liver 10 (FIG. 3). The probing instrument can be moved substantially along the predefined registration path to define a registration surface of the organ, at 155. For example, in some instances, the surgeon can move the probing instrument along the falciform ligament 11 of the liver 10 in the superior direction to the left triangular ligament 12 and subsequently to the right triangular ligament 13. As described with reference to FIG. 1, the probing instrument can include one or more IREDs that can be tracked by an optical tracking system. Thus, the registration path can be digitized and information associated with the registration path can be processed. Because the path of the instrument is predefined, the registration process is simplified as compared to a freeform registration process.

In some embodiments, the surface of the organ in physical space can be determined based at least in part on the registration path. For example, in some instances, the overall shape of the organ can be algorithmically defined. With at least a portion of the surface of the organ determined, the registration surface of the organ is mapped onto the image of the surface of the organ based at least in part on the registration path, at 156. For example, in some instances, the registration path in physical space is matched with the registration path on the image of the surface. In such instances, the initial matching of the registration paths can provide a starting point for an iterative mathematical matching of the surface of the organ in physical space (i.e., intraoperatively) to the image of the surface of the organ. For example, the matching of the registration paths can provide an initial matching for an iterative closet point (ICP) surface matching. By defining a starting point and at least a portion of the registration path associated with salient features of the organ, the process time for registering the surface of the organ intraoperatively to the image of the surface of the organ is reduced and the accuracy of the registration is increased. For example, by restricting the initial order of data collection, the registration of the surface of the organ to the image of the surface is biased towards starting point and/or the registration path at early iterations, while utilizing this initial alignment as an anchor at later iterations.

With the surface of the organ in physical space registered to the image of the organ, the position of a medical device (e.g., an ablation instrument or the like) can be tracked and graphically displayed (e.g., on the display 116 of the electronic processing device 110) on the image of the organ. Thus, the method 150 provides a means for image-guided intervention. Furthermore, the accuracy of the registration allows for a virtualization of the organ that is continually updated based on movement of the medical device.

The method 150 can be used to match an intraoperative surface of any suitable organ to a corresponding preoperative image. For example, FIG. 4 is an illustration of a pancreas 20. In such instances, a preoperative (preop) image of the pancreas 20 can be taken and a surgeon can identify a starting point associated with a registration path along the surface of the pancreas 20. For example, in some instances, the surgeon can define a starting point of the registration path at the pancreatic notch 21. The registration path can move along the surface of the pancreas 20 to the tail 22, the omental tuber 23, and around the duodenum 24. Thus, the registration path can be substantially followed along the surface of the pancreas 20 intraoperatively to define a registration surface. The registration surface in physical space can then be mapped to the image of the surface of the pancreas 20.

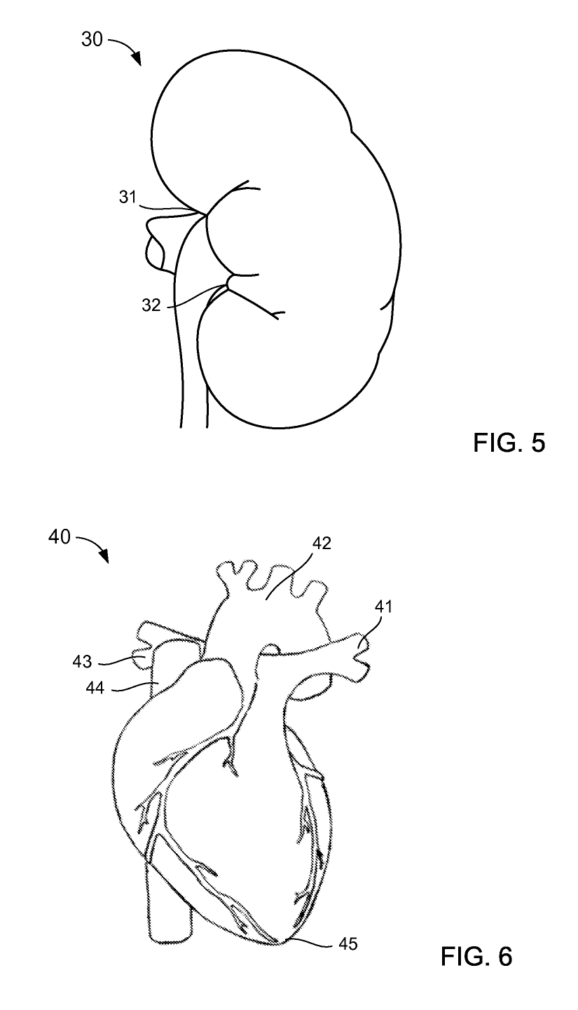

As shown in FIG. 5, the methods and embodiments described herein can be used to register a surface of a kidney 30. In some instances, a preoperative image of the kidney 30 can be taken and a surgeon can identify a starting point (e.g., a salient anatomical feature) associated with a registration path along the surface of the kidney 30. For example, in some instances, the surgeon can define a starting point of the registration path at the renal artery 31. The registration path can move along the surface of the kidney 30 to the ureter 32. Thus, the registration path can be substantially followed along the surface of the kidney 30 intraoperatively to define a registration surface. The registration surface in physical space can then be mapped to the image of the surface of the kidney 30.

As shown in FIG. 6, the methods and embodiments described herein can be used to register a surface of a heart 40. In some instances, a preoperative image of the heart 40 can be taken and a surgeon can identify a starting point (e.g., a salient anatomical feature) associated with a registration path along the surface of the heart 40. For example, in some instances, the surgeon can define a starting point of the registration path at the branch of the left pulmonary arteries 41. The registration path can move along the surface of the heart 40 around the aorta 42, the right pulmonary arteries 43, and the vena cava 44, to the tail 45 of the heart 40. Thus, the registration path can be substantially followed along the surface of the heart 40 intraoperatively to define a registration surface. The registration surface in physical space can then be mapped to the image of the surface of the heart 40.

While the methods and systems described above refer to matching an intraoperative surface of any suitable organ to a corresponding preoperative image, in some embodiments, the systems and methods described herein can be used to match an intraoperative surface of the skin of a patient to a preoperative image (e.g., from a CT scan, MRI, or the like). For example, in some instances, a portion of the abdomen can be scanned prior to an interventional procedure and a surface of the skin of the abdomen can be used to register anatomical features in physical space to the corresponding features in the preoperative scan. In some instances, abdomen surfaces can be used to register the anatomical features to the preoperative scan as described in U.S. Patent Publication No. 2011/0274324, entitled, “System and Method for Abdominal Surface Matching Using Pseudo-Features,” filed May 5, 2011, the disclosure of which is incorporated herein by reference in its entirety. In some instances, abdomen surfaces, organ surfaces, and/or pseudo-features (described in U.S. Patent Publication No. 2011/0274324) can be collectively used to register anatomical features to the preoperative scan.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above

Where schematics and/or embodiments described above indicate certain components arranged in certain orientations or positions, the arrangement of components may be modified. Similarly, where methods and/or events described above indicate certain events and/or procedures occurring in certain order, the ordering of certain events and/or procedures may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

What is claimed is:

1. A method, comprising:
scanning a bodily tissue of a patient with an imaging device and prior to an interventional procedure to produce an image of a surface of an organ, at least a portion of a registration path associated with the organ is defined;
surgically exposing the organ and placing a probing instrument in contact with the organ at a starting point associated with the registration path;
moving the probing instrument substantially along the registration path to define a registration surface of the organ; and
mapping the registration surface of the organ to the image of the surface of the organ based at least in part on the registration path.

2. The method of claim 1, wherein the image of the surface of the organ is displayed within a graphical user interface of a window manager during the mapping.
